(19) United States
(12) Patent Application Publication
(10) Pub. No.: US 2025/0235653 A1
(43) Pub. Date: Jul. 24, 2025

(54) COMFORT PAD FOR USE ON A CONTINUOUS POSITIVE AIRWAY PRESSURE FRAME

(71) Applicant: DCSTAR INC, New York, NY (US)

(72) Inventor: David LUO, New York, NY (US)

(73) Assignee: DCSTAR INC, New York, NY (US)

(21) Appl. No.: 18/416,188

(22) Filed: Jan. 18, 2024

(51) Int. Cl. A61M 16/06

(52) U.S. Cl. CPC A61M 16/0633; A61M 16/0683

(57) ABSTRACT

A comfort pad for use on a Continuous Positive Airway Pressure frame. The comfort pad is configured to contact a face and includes a left-right symmetric structure. The comfort pad includes a first surface that connects to the frame and a second surface that contacts the face, an upper side, a lower side, a left side, and a right side opposite the left side. The material of the comfort pad is either foam, fabric, or a combination of both to ensure a comfortable contact surface for patients.

### TECHNICAL FIELD

The disclosure pertains to the technical field of patient interface assemblies, specifically, the frame in the assembly involving sidebands or forehead support, and more specifically, it relates to the form and method of use of a comfort pad for use on a CPAP (Continuous Positive Airway Pressure) frame.

### BACKGROUND

Sleep apnea is a potentially dangerous disease, which not only causes fatigue in patients but also leads to a series of complications. Therefore, sleep apnea should be given adequate attention and can be treated by maintaining continuous positive airway pressure ventilation through a CPAP mask. Patients wear the CPAP mask over their nose and mouth during the night, with the mask connected to a ventilator by tubing. The ventilator pumps pressurized air into the patient's airway, keeping the collapsed airway open during sleep. However, due to the difficulty in satisfying the comfort needs of different patients with the various components of the patient interface assembly that contact the face, in actual use, up to 83% of patients do not comply with ventilator treatment. In the patient interface assembly, part of the frame is configured to have sidebands or a forehead support. The purpose of this design is to distribute the pressure on the patient's face of the patient interface assembly, thereby enhancing the patient's wearing experience. However, the increase in contact points also brings about issues of being comfortable or not where these points touch the patient's face. To address this issue, some patient interface assemblies, during production, directly connect fabric to the sidebands of the frame using heat pressing or adhesive methods. In some patient interface assemblies, the forehead support is combined with a silicone pad, all to provide a comfortable contact surface for the patient. The patient's face touching soft fabric or an elastic silicone pad can solve 80% of the indentation problems caused by rigid materials on the skin. However, due to the limited deformability of fabric or the non-breathability of silicone materials, it cannot be guaranteed that the patient's face will not be compressed under prolonged pressure, or that skin damage due to non-breathability can be avoided.

Therefore, the design of the frame in the patient interface assembly needs to consider not only whether its shape can distribute the pressure of the patient interface assembly on the patient's face, but also the comfort of its contact with the patient's face, to meet the comfort needs of different patients to the greatest extent possible.

### SUMMARY

Based on the background, it is necessary to address the aforementioned shortcomings and provide a comfort pad for use on a Continuous Positive Airway Pressure frame.

In one embodiment, a comfort pad is provided. The comfort pad is for use on a Continuous Positive Airway Pressure frame with sidebands, and the comfort pad including a left part and a right part that is symmetric to the left part. The comfort pad is configured to contact a face, and the comfort pad includes: a first surface that contacts the sidebands, a second surface that contacts the face, an upper side and a lower side. The comfort pad includes a first flexible material, which is configured to be a foam material, and is non-detachably connectable to the sidebands. The first surface, during use, has a contour similar to a shape of the sidebands.

In one embodiment, the comfort pad has a left side and a right side opposite the left side, the left side and the right side do not extend beyond connection points of the sidebands with other components, the other components are the frame or the headband.

In one embodiment, a connection method between the comfort pad and the sidebands is one of the following methods: adhesive, heat pressing, welding, ultrasonic, or sewing.

In one embodiment, a contour of the first surface, which is similar to the shape of the sidebands, is one of the following shapes: straight line-shaped, curved line-shaped, Y-shaped, V-shaped, or T-shaped.

In another embodiment, a comfort pad is provided. The comfort pad is for use on a Continuous Positive Airway Pressure frame with sidebands, and the comfort pad including a left part and a right part that is symmetric to the left part. The comfort pad is configured to contact a face. The comfort pad includes: a first surface that contacts the sidebands, a second surface that contacts the face, an upper side and a lower side. The comfort pad includes a first flexible material, which is configured to be a foam material, and is detachably connectable to the sidebands. The first surface, during use, has a contour similar to a shape of the sidebands.

In one embodiment, the comfort pad has a left side and a right side opposite the left side, the left side and the right side do not extend beyond connection points of the sidebands with other components, the other components are either the frame or the headband.

In an embodiment, a connection method between the comfort pad and the sidebands is one of the following methods: a double-sided tape, a peelable glue, a hook and loop fastener, a flexible magnet, a snap, or a strap.

In an embodiment, a contour of the first surface, which is similar to the shape of the sidebands, is one of the following shapes: straight line-shaped, curved line-shaped, Y-shaped, V-shaped, or T-shaped.

In another embodiment, a comfort pad is provided. The comfort pad is for use on a Continuous Positive Airway Pressure frame. The comfort pad has sidebands and includes a left part and a right part that is symmetric to the left part. The comfort pad is configured to contact a face. The comfort pad includes: a first surface that contacts the sidebands, a second surface that contacts the face, an upper side and a lower side. The comfort pad includes a first flexible material, which is configured to be a foam material. Part of a contour of the comfort pad is larger than a contour of the sidebands. The first surface, during use, has a contour similar to a shape of the sidebands. The upper side and the lower side which connect the first surface and the second surface of the comfort pad have an arcuate surface.

In an embodiment, the comfort pad has a left side and a right side opposite the left side, the left side and the right side do not extend beyond connection points of the sidebands with other components, and the other components are either the frame or the headband.

In an embodiment, the arcuate surfaces of the upper side and the lower side are rounded.

In an embodiment, a width of a part of the comfort pad that is larger than a contour of the sidebands does not exceed 10 mm.

In another embodiment, a comfort pad is provided. The comfort pad is for use on a Continuous Positive Airway Pressure frame. The comfort pad is left-right symmetric and is configured to contact a face. The comfort pad includes: a first surface that contacts the frame, a second surface that contacts the face, an upper side and a lower side. The comfort pad includes a first flexible material, which is configured to be a foam material. The first surface, during use, has a contour similar to a shape of the sidebands or a forehead support. The foam material has one or more of the following characteristics:
a. a thickness at or between 2 to 20 mm;
b. a density of at least 30 kg/m³;
c. a tear strength of at least 1.5 N/cm; and
d. a tensile strength of at least 5 N/cm².

In an embodiment, the comfort pad has a left side and a right side opposite the left side, the left side and the right side do not extend beyond connection points of the sidebands with other components, and the other components are either the frame or the headband.

In an embodiment, a contour of the first surface, which is similar to the shape of the sidebands, is one of the following shapes: straight line-shaped, curved line-shaped, Y-shaped, V-shaped, or T-shaped.

In an embodiment, a part of the frame connectable to the comfort pad is either the sidebands or the forehead support.

In another embodiment, a comfort pad is provided. The comfort pad is for use on a Continuous Positive Airway Pressure frame. The comfort pad is left-right symmetric and is configured to contact a face. The comfort pad includes: a first surface that contacts the frame, a second surface that contacts the face, and an upper side and a lower side. The comfort pad is configured to be detachably connectable to the frame and includes a first flexible material and a second flexible material, with the second flexible material connectable to the first flexible material. The first surface, during use, has a contour similar to a shape of the sidebands or a forehead support.

In an embodiment, the comfort pad has a left side and a right side opposite the left side, the left side and the right side do not extend beyond connection points of the sidebands with other components, and the other components are either the frame or the headband.

In an embodiment, the first flexible material is a foam material, and the second flexible material is a fabric material.

In an embodiment, a contour of the first surface, which is similar to the shape of the sidebands, is one of the following shapes: straight line-shaped, curved line-shaped, Y-shaped, V-shaped, or T-shaped.

In an embodiment, a part of the frame connectable to the comfort pad is either the sidebands or the forehead support.

Implementing the comfort pad for use on the Continuous Positive Airway Pressure frame of this disclosure has at least the following beneficial effects:

1. Increasing Comfort: Some patient interface assemblies available on the market, due to stability requirements, have frames with sidebands or forehead supports. Sidebands and forehead support, to provide better stability for the frame, typically prefer rigid materials, such as polycarbonate or polyethylene. However, the rigid materials on the sidebands can also create discomfort for patients: patients often wear these for long periods at night, and cannot control their sleeping posture once asleep. This can lead to, with great possiblities, red marks or pressure sores due to the sidebands pressing on the face when lying on one side. To address this issue, some patient interface assemblies, during production, directly connect fabric to the sidebands of the frame using heat pressing or adhesive methods. Some patient interface assemblies combine the forehead support with a silicone pad, all aiming to provide a comfortable contact surface for the patient. The patient's face touching soft fabric or an elastic silicone pad can solve 80% of the indentation problems caused by rigid materials on the skin. However, due to the limited deformability of fabric or the non-breathability of silicone materials, it cannot be guaranteed that the patient's face will not be compressed under prolonged pressure, or that skin damage due to non-breathability can be avoided. According to market data analysis, half of the users who purchase frames with fabric sidebands choose to buy an additional pair of comfort covers for overlaid use. Therefore, this dislcosure selects foam material, which has a stronger deformation capacity and better breathability compared to fabric, to connect with the sidebands of the frame and forehead support. a. The foam material is elastic, and its porous structure expands under external pressure, dispersing the pressure over a broader area, reducing the feeling of compression in the sideband and forehead support areas, and better dispersing pressure. b. The soft texture of the foam material allows it to better adapt to the three-dimensional curves of the human face, air can circulate through the pores, keeping the foam material fluffy and not pressing against the face, also avoiding skin allergies like pressure sores and red marks caused by the non-breathability of rigid materials. c. When the patient does not use it during the day, the foam material will return to its original shape due to its rebound characteristics, ready for the next use by the patient. d. Due to its interstitial spaces and surface tension, the foam material's interstitial spaces can accommodate gas and liquid molecules. When sweat and oil adhere to the foam material, it draws in gas or liquid molecules, maintaining dry and comfortable skin for the patient throughout the night, and preventing sidebands from relative displacement with the patient's face due to sweat or oil secretions. Since the patient's face connects with the frame through the foam material and does not directly contact the frame, it effectively prevents secretions or friction from the patient's face from damaging the sideband, thereby enhancing the lifespan of the frame.

2. Simpler Process: Some frames on the market use fabric combined with foam material to enhance overall deformability, but the composite process is often cumbersome and complex: a. Select a base material to become the foam body and prepare the fabric. b. Slice the material into pieces to achieve the desired thickness. c. Clean the surfaces of the foam material and fabric. d. Choose a suitable adhesive (ensuring that the adhesive is compatible with the materials being bonded). e. Adhesion (apply adhesive to one of the material surfaces, ensure a tight fit between the two, place the composite material into a press or fixture for pressure application, then dry and cure). f. Inspection and repair (check for complete bonding, any bubbles, or insecure areas). g. Cut into the desired shape. Since composite materials are usually two or more different materials with different textures and properties, some also involve edging processes to prevent delamination, peeling, or wear during use, thereby enhancing product durability and lifespan. After obtaining the comfort pad with composite material, it is then connected to the frame through heat pressing or adhesive. The preparation steps for the comfort pad material in this disclosure are relatively simple (only requiring the preparation of foam material or fabric): a. Select the base material to become the foam body. b. Slice the material into pieces to achieve the desired thickness. c. Cut into the required shape using a CNC cutting process, or involve fabric in sewing and cutting. After obtaining the desired shape of the comfort pad, connect it to the frame using adhesive, double-sided tape, or other methods, reducing the difficulty in making the comfort pad.

3. Detachable Replacement: Frames with sidebands on the market that also have fabric combined with foam material are all in a non-detachable form. Patients, unable to dismantle them, can only clean away surface stains. However, due to the numerous tiny pores and fiber structures in the foam material, which can firmly absorb liquids inside through capillary action, it is not easy to completely separate the stains and foam material with water or detergents. Therefore, even if the surface looks clean, residual oil stains may still exist. Taking advantage of the easily obtainable characteristics of foam material, the disclosure provides a comfort pad with a detachable feature. The foam material can be detachably connectable to the sidebands of the frame and forehead support through a double-sided tape, a peelable glue, a hook and loop fastener, or via structural design. Alternatively, fabric can be sewn into a form that can accommodate foam material and be detachably connectable to the sidebands of the frame and forehead support. The detachable and replaceable comfort pad ensures cleanliness and hygiene for the sidebands and forehead support, preventing discomfort from accumulated oil stains for the patient. Moreover, the detachable replacement of comfort pad is more environmentally friendly. Patients only need to remove and dispose of the foam material, without the need to replace the entire frame, reducing the increase in carbon emissions caused by waste.

### BRIEF DESCRIPTION OF THE DRAWINGS

FIG. 1 is a schematic diagram of the structure of the comfort pad on the sidebands in accordance with an embodiment;

FIG. 2 is a schematic diagram of the structure of the comfort pad on the forehead support in accordance with an embodiment;

FIG. 3 is a schematic diagram of the region of the comfort pad in accordance with an embodiment;

FIG. 4A and FIG. 4B are planar area schematic diagrams of the comfort pad in accordance with an embodiment;

FIG. 5A, FIG. 5B and FIG. 5C are schematic diagrams of different forms of the comfort pad (corresponding to sidebands) in accordance with an embodiment;

FIG. 6A, FIG. 6B and FIG. 6C are schematic diagrams of different forms of the comfort pad (corresponding to forehead support) in accordance with an embodiment;

FIG. 7A, FIG. 7B, FIG. 7C, and FIG. 7D are schematic diagrams of the non-detachable connection method of the comfort pad in accordance with an embodiment;

FIG. 8A and FIG. 8B are schematic diagrams of different forms of the upper side and lower side of the comfort pad in accordance with an embodiment;

FIG. 9A and FIG. 9B are planar schematic diagrams of the distance by which the comfort pad extends beyond the frame in accordance with an embodiment;

FIG. 10A, FIG. 10B and FIG. 10C are schematic diagrams of the cross-section of different forms of the comfort pad in accordance with an embodiment;

FIG. 11 is a schematic diagram of different forms of the detachable connection method of the comfort pad in accordance with an embodiment;

FIG. 12A and FIG. 12B are partial enlarged schematic diagrams of different connection methods of “D” in FIG. 11;

FIG. 13A and FIG. 13B are schematic diagrams of different forms of the detachable structure of the comfort pad in accordance with an embodiment;

FIG. 14 is a schematic diagram of the cross-sectional structure of the comfort pad in accordance with an embodiment;

FIG. 15 is a schematic diagram of different forms of the detachable structure of the comfort pad in accordance with an embodiment;

FIG. 16 is a schematic diagram of the connection between the first flexible material and the second flexible material of the comfort pad in accordance with an embodiment;

FIG. 17 is a schematic diagram of another connection method between the first flexible material and the second flexible material of the comfort pad in accordance with an embodiment;

FIG. 18 is a regional division diagram of the left and right parts in accordance with several embodiments.

### DETAILED DESCRIPTION

To make the purposes, features, and advantages of a connector for a frame of a respirator clearer and more understandable, the specific embodiments of this disclosure are detailed below, accompanied by illustrations. Many specific details are explained in the following descriptions to ensure a thorough understanding of the disclosure. However, the disclosure can be implemented in so many ways that are different from the ones described here, and professionals in the field can make modifications without departing from the spirit of the disclosure. Therefore, the disclosure is not limited to the specific embodiment disclosed below.

This disclosure aims to address the lack of comfort in the sidebands or forehead supports of the existing patient interface assemblies. They, during patients' prolonged night-time wear, lead to issues such as red marks and pressure sores, which are formed by the sidebands pressing against the face because of lying on their side. The non-breathable nature of the silicone pads in the forehead supports causes oil and sweat accumulation on the patient's forehead skin, which cannot be discharged in time and therefore causes damage to the product. Additionally, some patient interface assemblies have non-detachable comfort pads, preventing patients from replacing them with clean ones. Therefore, a comfort pad for use on a Continuous Positive Airway Pressure frame is provided. This comfort pad is made of materials with greater deformability and better breathability. Its structural design allows for detachable or non-detachable connections with the frame (such as the sidebands on the frame), offering patients more comfortable options. The frame with the comfort pad can deform according to the contours of the patient's face, reducing displacement with the face, thus making the connection between the patient interface assembly and the patient's face more stable. Additionally, frames with sidebands or forehead supports can distribute the pressure on the patient's face from the assembly through the comfort pad, making the patient's wearing more comfortable, easing their mindset during treatment, and leading to better compliance.

The following embodiments illustrate several forms of the comfort pad used for a Continuous Positive Airway Pressure frame according to this disclosure.

### Embodiment 1

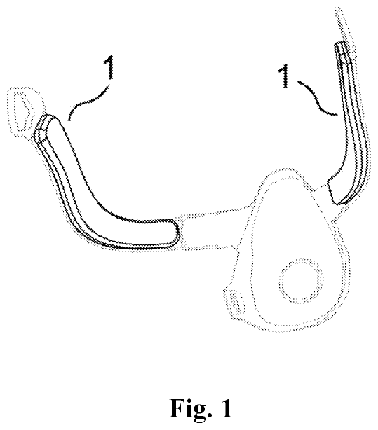

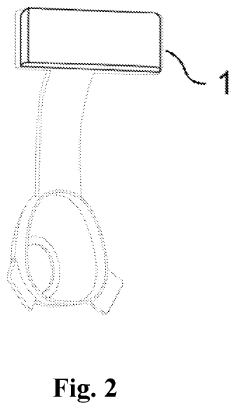

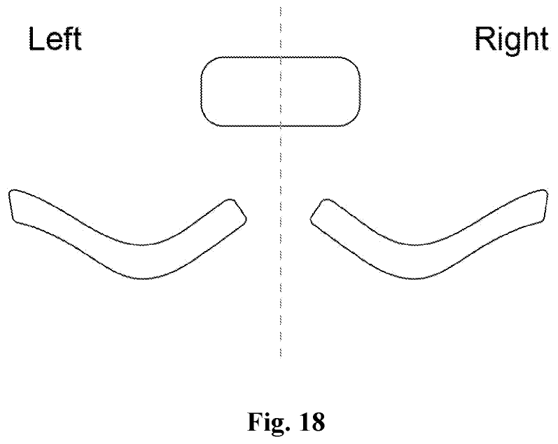

The objective of this disclosure is to provide a comfort pad 1 for use on a Continuous Positive Airway Pressure frame, as shown in FIGS. 1-2 and 18. This comfort pad 1 is configured to have a left part and a right part that is symmetrical to the left part, configured to partially contact the patient's face and enhance the fixation of the overall patient interface assembly with the patient's face, thereby better distributing pressure.

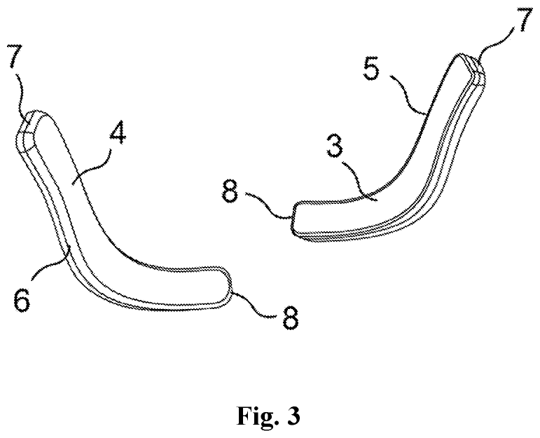

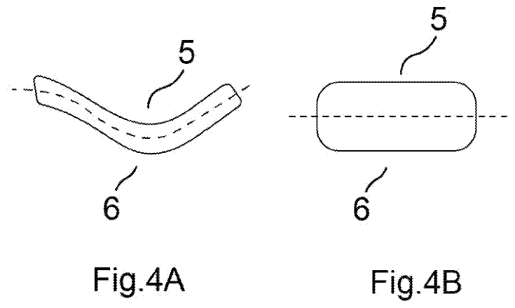

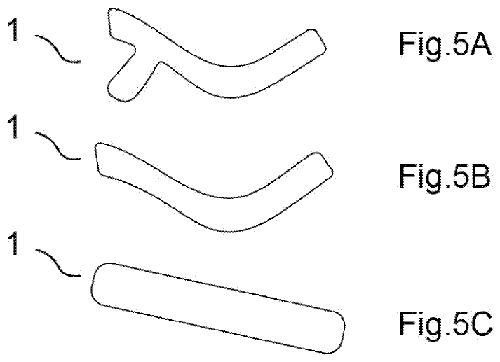

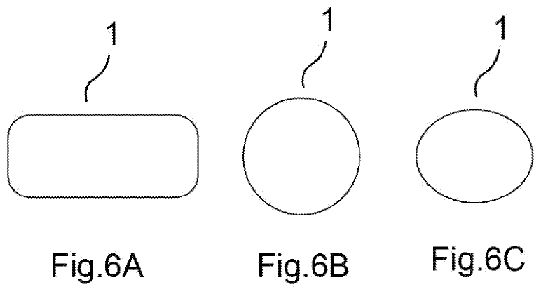

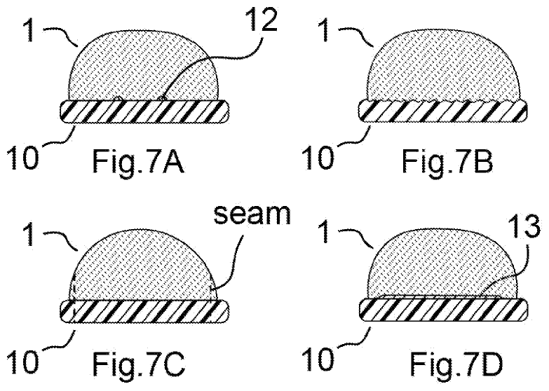

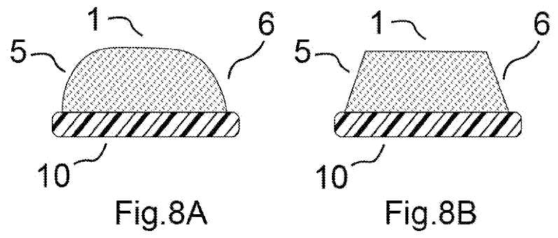

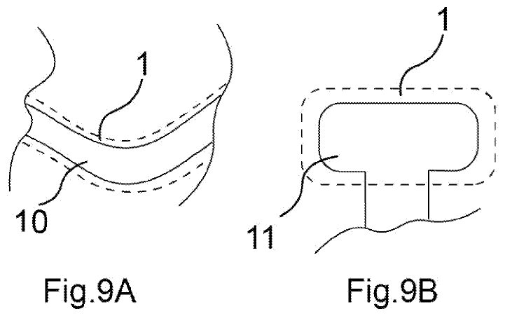

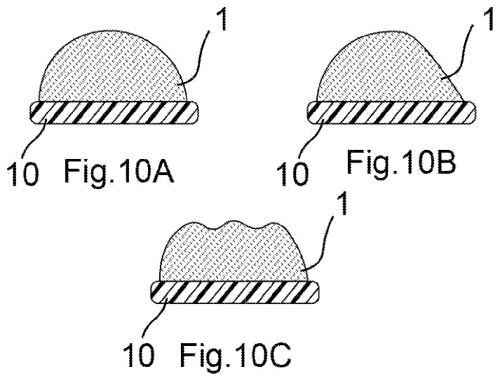

Specifically, as shown in FIG. 3, the figure displays the front and back of a single-sided comfort pad 1. The comfort pad 1 has a first surface 3 that contacts the sidebands 10 or forehead support 11, a second surface 4 that contacts the patient's face, an upper side 5 and a lower side 6 (as shown in FIG. 4, the upper side 5 and lower side 6 are determined by the comfort pad 1 itself; when the comfort pad 1 is placed on the frame in the corresponding position, the midline distinguishes the upper side 5 and the lower side 6: the part above the midline of the comfort pad 1 is the upper side 5, and the part below is the lower side 6). The comfort pad 1 has a left side 7 and a right side 8 opposite the left side 7, and the left side 7 and right side 8 do not extend beyond the connection points of the sidebands 10 with other components (including the frame, headband, etc.); the first surface 3 of the comfort pad 1 has a contour similar to the shape of the sidebands 10 or forehead support 11. As shown in FIG. 5, the shape contour corresponding to the sidebands 10 can be straight line-shaped, curved line-shaped, Y-shaped, V-shaped, or T-shaped, etc. As shown in FIG. 6, the shape contour corresponding to the forehead support 11 can be circular, square, elliptical, or other symmetrical shapes. As shown in FIG. 10, the cross-sectional shape of the comfort pad 1 can be semi-circular, wavy, triangular, etc. Additionally, as shown in FIG. 9, part of the contour of the comfort pad 1 can exceed that of the sidebands 10 (FIG. 9A) or forehead support 11 (FIG. 9B), but the distance between the two contours does not exceed 10 mm, to prevent the comfort pad 1 from losing the support of the sidebands 10 and easily wrinkling or deforming, affecting the patient's wearing experience. As shown in FIG. 8A, the upper side 5 and lower side 6 of the comfort pad 1 have an arcuate surface. The form of the surface can be equidistant rounded corners or non-equidistant rounded corners, creating a smooth contact surface to provide a comfortable contact experience for the patient's face. The thickness of the comfort pad 1 is at or between 2 to 20 mm, ensuring sufficient deformation to support and fit the patient's face. The comfort pad 1 is made from foam material that is both deformable and breathable. The foam material can be made from wood cellulose or foamed plastic polymers, or synthetic sponges such as polyvinyl alcohol (a highly absorbent material with no obvious pores), polyester, or polyurethane, etc. To ensure the comfort pad 1 made from foam material is durable and comfortable, the foam should have one or more of the following characteristics: a. a density of approximately 30 kg/m³, b. a tear strength of at least 1.5 N/cm (tested according to ASTM (American Society for Testing and Materials) D412-16), and c. a tensile strength of at least 5 N/cm² (tested according to ASTM D624-00). In this embodiment, as shown in FIG. 7, the connection between the comfort pad 1 and the frame (such as the sidebands 10 or forehead support 11 on the frame, with FIG. 7 showing only the connection between the comfort pad 1 and the sidebands 10) is a non-detachable connection, which can be a direct connection achieved through adhesive 12, heat pressing, welding, ultrasonic, or sewing, or connected through a thin film 13 made of thermoplastic material. The adhesive 12 is usually glue, and to ensure the performance between the foam material, frame, and adhesive 12, and to prevent the adhesive 12 from damaging the appearance and characteristics of the foam material or frame, becoming hard or whitening after curing, attention should be paid to the following aspects when it comes to choosing the adhesive 12: a. Adhesive strength, to ensure that the connection between the frame and the comfort pad 1 is firm enough. The usage of adhesive 12 should be at least 0.03 g, with a density at or between 0.3 to 3 g/cm³, a temperature resistance range at or between −55° C. to 200° C., and a tensile strength of at least 0.5 MPa. This ensures that the connection force between the frame and comfort pad 1, without any external force applied, is greater than the gravitational force acting on the comfort pad 1 itself and there will be no detachment because of movement during sleep or wrinkles causing leaks; at the same time, the adhesive strength should not be too strong to avoid residue during recycling and disassembly, or damage to the materials. b. Material Characteristics: The adhesive 12, which is located near the patient's respiratory tract and face when the product is in use, should meet basic non-toxic, odorless, and other harmless-to-human characteristics. After curing, it should be transparent or semi-transparent to avoid leaving marks that affect the final appearance of the frame. It is essential to ensure the compatibility and adaptability of the bonding materials to avoid delamination, surface damage, or penetration during the bonding process. The adhesive 12 must be non-toxic and adaptable, and comply with local regulatory requirements, such as the ISO 10993 standard. When used, the adhesive 12 should be in a liquid or semi-flowing state. After curing and setting, the hardness should be greater than or equal to Shore A 15, and the tensile strength should be greater than or equal to 2 N/cm², to prevent it from being too hard and exerting pressure on the patient's face or causing stiffness and discomfort. c. Environmental Consideration: The adhesive 12 should be chosen with the one with longevity to avoid rapid aging of the product. Preference should be given to environmentally harmless and biodegradable adhesives 12, with considerations for energy saving, waste disposal, and process optimization. Brands such as 3M, Loctite, UHU, and the like could be considered for the glue.

In another embodiment, as shown in FIG. 8B, the upper side 5 and lower side 6 of the comfort pad 1 are straight surfaces (or inclined surfaces).

### Embodiment 2

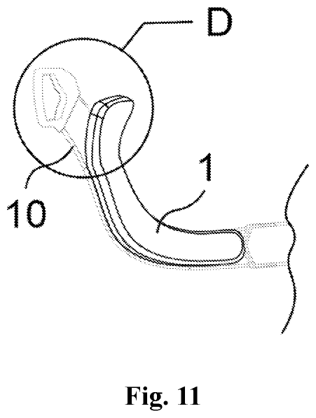

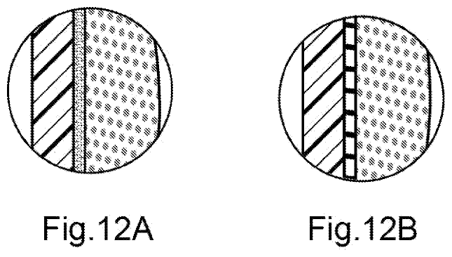

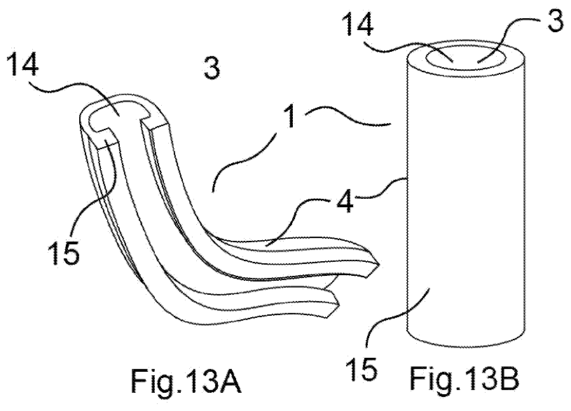

The objective of this disclosure is to provide a comfort pad 1 for use on a Continuous Positive Airway Pressure frame. This comfort pad 1 is configured to have a left part and a right part that is symmetrical to the left part, configured to partially contact the patient's face and enhance the fixation of the overall patient interface assembly with the patient's face, thereby better distributing pressure. The comfort pad 1 includes a first surface 3 that contacts the sidebands 10 or forehead support 11, a second surface 4 that contacts the face, an upper side 5 and a lower side 6. The comfort pad has a left side 7 and a right side 8 opposite the left side. This embodiment provides a comfort pad 1 for use on a Continuous Positive Airway Pressure frame that differs from the comfort pad 1 in Embodiment 1 in that the connection between the comfort pad 1 and the frame is detachable. As shown in FIGS. 11 and 12, the detachable connection methods can include a double-sided tape, a peelable glue, a hook and loop fastener (as shown in FIG. 12A), a flexible magnet (as shown in FIG. 12B), a snap, a strap, etc. As shown in FIG. 13A, the comfort pad 1 can also be detachably connectable to the frame through its own shape and contour design. Specifically, the comfort pad 1 has a chamber 14 to accommodate the sidebands 10 or forehead support 11, and a fixing part 15 that contacts at least one side of the sidebands 10 or forehead support 11, configured to fix the comfort pad 1 onto the sidebands 10 or forehead support 11. As shown in FIG. 13B, the comfort pad 1 itself can be detachably connectable to the frame. Specifically, the comfort pad 1 has a chamber 14 to accommodate the sidebands 10 or forehead support 11, and the comfort pad 1 is directly connectable to the sidebands of the frame or forehead support 11 through a closed annular connection.

### Embodiment 3

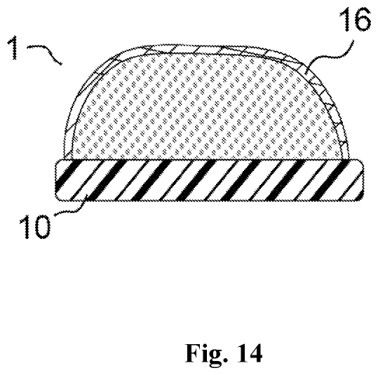

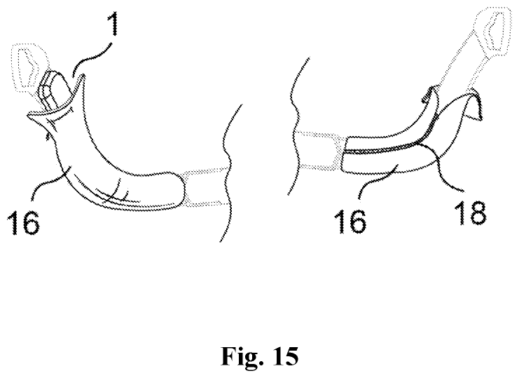

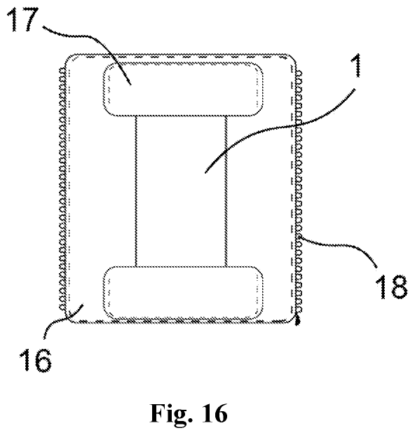

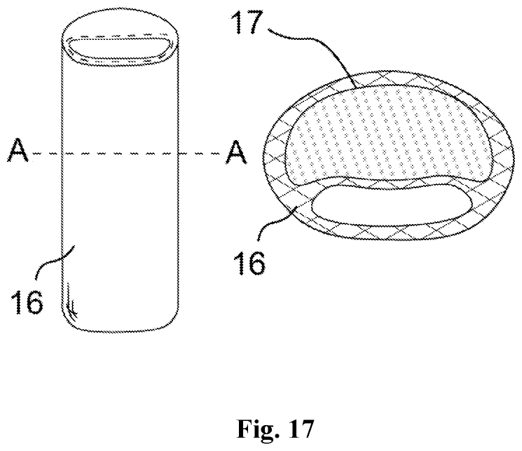

The objective of this disclosure is to provide a comfort pad 1 for use on a Continuous Positive Airway Pressure frame. This comfort pad 1 is configured to have a left part and a right part that is symmetrical to the left part, configured to partially contact the patient's face and enhance the fixation of the overall patient interface assembly with the patient's face, thereby better distributing pressure. The comfort pad 1 includes a first surface 3 that contacts the sidebands 10 or forehead support 11, a second surface 4 that contacts the face, an upper side 5 and a lower side 6. The comfort pad has a left side 7 and a right side 8 opposite the left side. This embodiment provides a comfort pad 1 for use on a Continuous Positive Airway Pressure frame that differs from the comfort pad 1 in Embodiment 1 in that: the composition material of the comfort pad 1 is different, and the connection between the comfort pad 1 and the frame is detachable. As shown in FIG. 14, this comfort pad 1 includes a first flexible material and a second flexible material, with the second flexible material being connectable to the first flexible material. The first flexible material can be foam, and the second flexible material can be fabric 16. The first flexible material has the same characteristics as the foam material in Embodiment 1, and the second material is fabric 16, which is highly absorbent and breathable. It can effectively absorb the patient's facial secretions at night, keeping the patient's face dry and breathable, and improving the patient's compliance with the patient interface pad. The composition of the fabric 16 includes one or several materials such as cotton, silk, velvet, nylon, chinlon, spandex, polyester, etc., made in various proportions. Some of the fabric 16 can also be made with metal fibers to create different functional fabrics 16, such as antibacterial fabric, antistatic fabric, and so on. Fabric 16 should have a permeability at or between 1 to 30 CFM (determined by the air permeability test method in ASTM D737). In another embodiment, the air permeability is at or between 40 to 60 CFM, or 50 to 80 CFM, ensuring the patient's comfort while avoiding excessive gas leakage. Fabric 16 should have a water absorption time of less than 30 seconds, preferably less than 20 seconds, and more ideally less than 10 seconds, to ensure it can absorb the patient's facial sweat and oils. The yarn count of fabric 16 should be no less than 30 and no more than 100, and the average roughness (Ra value) of the second surface 4 that contacts the patient's face should be at or between 0.2 to 10 micrometers, ensuring fabric 16 provides a soft and comfortable surface for the patient, while avoiding excessive weight of fabric 16 that could compress the patient's face. To ensure the comfort of the patient interface pad, the thickness of fabric 16 should be approximately at or between 0.8 mm to 3.5 mm (fabric 16 layers that are too thin easily lose elasticity and softness, whereas too thick fabric 16 layers are prone to displacement and leakage). The thickness of the fabric 16 can be uniform or varied, and it can be in a single layer or multiple layers. The combination of multiple layers of fabric 16 can be achieved by lamination, sewing, thermoplastic composites, or hot melt composites, and film 13 composites. The connection between fabric 16 and foam material can be a composite connection or a regular connection. Specifically, when fabric 16 and foam material are composite-connected, the first surface 3 of the comfort pad 1 that contacts the frame is made of foam material, and the second surface 4 that contacts the patient is made of fabric 16. Alternatively, by sewing fabric 16, it can have a shape contour similar to the sidebands 10 or forehead support 11 of the frame during use. The first surface 3 of the comfort pad 1 contacting the frame is made of fabric 16 and the second surface 4 of the comfort pad 1 contacting the patient is made of fabric 16 too. As shown in FIGS. 15-17, when fabric 16 and foam material are not connected through adhesive, the first surface 3 of the comfort pad 1 contacting the frame can be fabric 16, and the second surface 4 contacting the patient can be either fabric 16 or foam material. Specifically, fabric 16 can be cut and sewn, configured to be a chamber 17 to accommodate the foam material, as well as a fixing part 18 for connecting to the sidebands 10 (shown as a zipper in the figures, but other forms are possible in other embodiments). Openings can be made in fabric 16 to expose the foam material for direct contact with the patient's face. Through this method, the foam material and fabric 16 can be detachably connectable, allowing for timely replacement.

Implementing the comfort pad for use on the CAPA frame of this disclosure has at least the following beneficial effects:

1. Increasing Comfort: Some patient interface assemblies available on the market, due to stability requirements, have frames with sidebands or forehead supports. Sidebands and forehead support, to provide better stability for the frame, typically prefer rigid materials, such as polycarbonate or polyethylene. However, the rigid materials on the sidebands can also create discomfort for patients: patients often wear these for long periods at night, and cannot control their sleeping posture once asleep. This can lead to, with great possiblities, red marks or pressure sores due to the sidebands pressing on the face when lying on one side. To address this issue, some patient interface assemblies, during production, directly connect fabric to the sidebands of the frame using heat pressing or adhesive methods. Some patient interface assemblies combine the forehead support with a silicone pad, all aiming to provide a comfortable contact surface for the patient. The patient's face touching soft fabric or an elastic silicone pad can solve 80% of the indentation problems caused by rigid materials on the skin. However, due to the limited deformability of fabric or the non-breathability of silicone materials, it cannot be guaranteed that the patient's face will not be compressed under prolonged pressure, or that skin damage due to non-breathability can be avoided. According to market data analysis, half of the users who purchase frames with fabric sidebands choose to buy an additional pair of comfort covers for overlaid use. Therefore, this dislcosure selects foam material, which has a stronger deformation capacity and better breathability compared to fabric, to connect with the sidebands of the frame and forehead support. a. The foam material is elastic, and its porous structure expands under external pressure, dispersing the pressure over a broader area, reducing the feeling of compression in the sideband and forehead support areas, and better dispersing pressure. b. The soft texture of the foam material allows it to better adapt to the three-dimensional curves of the human face, air can circulate through the pores, keeping the foam material fluffy and not pressing against the face, also avoiding skin allergies like pressure sores and red marks caused by the non-breathability of rigid materials. c. When the patient does not use it during the day, the foam material will return to its original shape due to its rebound characteristics, ready for the next use by the patient. d. Due to its interstitial spaces and surface tension, the foam material's interstitial spaces can accommodate gas and liquid molecules. When sweat and oil adhere to the foam material, it draws in gas or liquid molecules, maintaining dry and comfortable skin for the patient throughout the night, and preventing sidebands from relative displacement with the patient's face due to sweat or oil secretions. Since the patient's face connects with the frame through the foam material and does not directly contact the frame, it effectively prevents secretions or friction from the patient's face from damaging the sideband, thereby enhancing the lifespan of the frame.

2. Simpler Process: Some frames on the market use fabric combined with foam material to enhance overall deformability, but the composite process is often cumbersome and complex: a. Select a base material to become the foam body and prepare the fabric. b. Slice the material into pieces to achieve the desired thickness . . . c. Clean the surfaces of the foam material and fabric. d. Choose a suitable adhesive (ensuring that the adhesive is compatible with the materials being bonded). e. Adhesion (apply adhesive to one of the material surfaces, ensure a tight fit between the two, place the composite material into a press or fixture for pressure application, then dry and cure). f. Inspection and repair (check for complete bonding, any bubbles, or insecure areas). g. Cut into the desired shape. Since composite materials are usually two or more different materials with different textures and properties, some also involve edging processes to prevent delamination, peeling, or wear during use, thereby enhancing product durability and lifespan. After obtaining the comfort pad with composite material, it is then connected to the frame through heat pressing or adhesive. The preparation steps for the comfort pad material in this disclosure are relatively simple (only requiring the preparation of foam material or fabric): a. Select the base material to become the foam body. b. Slice the material into pieces to achieve the desired thickness. Cut into the required shape using a circular cutting process, or involve fabric in sewing and cutting. After obtaining the desired shape of comfort pad, connect it to the frame using adhesive, double-sided tape, or other methods, significantly reducing the difficulty in making the comfort pad.

3. Detachable and Replaceable: Frames with sidebands on the market that also have fabric combined with foam material are all in a non-detachable form. Patients, unable to dismantle them, can only clean away surface stains. However, due to the numerous tiny pores and fiber structures in the foam material, which can firmly absorb liquids inside through capillary action, it is not easy to completely separate the stains and foam material with water or detergents. Therefore, even if the surface looks clean, residual oil stains may still exist. Taking advantage of the easily obtainable characteristics of foam material, the disclosure provides a comfort pad with a detachable feature. The foam material can be detachably connectable to the sidebands of the frame and forehead support through a double-sided tape, a peelable glue, a hook and loop fastener, or via structural design. Alternatively, fabric can be sewn into a form that can accommodate foam material and be detachably connectable to the sidebands of the frame and forehead support. The detachable and replaceable comfort pad ensures cleanliness and hygiene for the sidebands and forehead support, preventing discomfort from accumulated oil stains for the patient. Moreover, the detachable replacement of comfort pad is more environmentally friendly. Patients only need to remove and dispose of the foam material, without the need to replace the entire frame, reducing the increase in carbon emissions caused by waste.

The technical features of the embodiments described above can be combined in any manner. To keep the description concise, not all possible combinations of the technical features in the above embodiments are described. However, as long as there is no contradiction in these combinations of technical features, they should be considered within the scope of this specification.

The embodiments described above only represent a few of the possible implementations of this disclosure and are described in specific and detailed terms, but they should not be construed as limiting the scope of the patent for the disclosure. It should be noted that, for those skilled in the art, various modifications and improvements can be made without departing from the concept of this disclosure, and these are also within the scope of protection of this disclosure. Therefore, the scope of protection of the patent for this disclosure should be determined by the attached claims.

It must be noted that as used herein and in the appended claims, the singular forms “a”, “an”, and “the” include their plural equivalents, unless the context clearly dictates otherwise.

1. A comfort pad, for use on a Continuous Positive Airway Pressure frame with sidebands, the comfort pad comprising:
a left part and a right part that is symmetric to the left part, wherein the comfort pad is configured to contact a face, and wherein each of the left part and right part comprises:
an inside surface that is configured to contact the face, an outside surface opposite the inside surface, an upper side, and a lower side;
a first flexible material having a first chamber and a second chamber; and
a foam material in the first chamber,
wherein the second chamber is empty, and
wherein an average roughness of the inside surface is less than or equal to 10 micrometers.

2. The comfort pad according to claim 1, wherein each of the left part and right part includes a left side and a right side opposite the left side, the left side and the right side do not extend beyond connection points of the sidebands with other components, and the other components include the frame or a headband.

3. (canceled)

4. The comfort pad according to claim 1, wherein the contour of the inside surface, which is similar to the shape of the sidebands, includes one of the following shapes: straight line-shaped, curved line-shaped, Y-shaped, V-shaped, or T-shaped.

5. A comfort pad, for use on a Continuous Positive Airway Pressure frame with sidebands, and the comfort pad comprising a left part and a right part that is symmetric to the left part, wherein the comfort pad is configured to contact a face, and wherein each of the left part and right part comprises:
a body made of foam material having an interior space that forms a chamber configured to accommodate part of the frame, the body having an inside surface that is configured to contact the face, an outside surface opposite the inside surface, an upper side and a lower side,
wherein the foam material has a thickness between 2 mm to 20 mm.

6. The comfort pad according to claim 5, wherein each of the left part and right part includes a left side and a right side opposite the left side, the left side and the right side do not extend beyond connection points of the sidebands with other components, and the other components include the frame or a headband.

7. The comfort pad according to claim 5, wherein the upper side and the lower side are rounded.

8. The comfort pad according to claim 5, wherein a width of a part of the comfort pad that is larger than the contour of the sidebands does not exceed 10 mm.

9. (canceled)

10. The comfort pad according to claim 5, wherein the contour of the inside surface, which is similar to the shape of the sidebands, is one of the following shapes: straight line-shaped, curved line-shaped, Y-shaped, V-shaped, or T-shaped.

11. (canceled)

12. A comfort pad, for use on a Continuous Positive Airway Pressure frame, which has a left part and a right part symmetric to the left part, wherein the comfort pad is configured to contact a face, and wherein each of the left part and right part comprises:
an inside surface that is configured to contact the face and an outside surface opposite the inside surface;
an upper side and a lower side;
wherein the comfort pad includes a first flexible material, which is configured to be a foam material;
wherein the first surface, during use, includes a contour similar to a shape of sidebands or a forehead support; and
wherein the foam material has a thickness between 2 mm to 20 mm.

13. The comfort pad according to claim 12, wherein the comfort pad includes a left side and a right side opposite the left side, the left side and the right side do not extend beyond connection points of the sidebands with other components, and the other components are the frame or a headband.

14. The comfort pad according to claim 12, wherein the contour of the first surface, which is similar to the shape of the sidebands, includes one of the following shapes: straight line-shaped, curved line-shaped, Y-shaped, V-shaped, or T-shaped.

15. The comfort pad according to claim 12, wherein a part of the frame connectable to the comfort pad includes the sidebands or the forehead support.

16. A comfort pad, for use on a Continuous Positive Airway Pressure frame, wherein the comfort pad is configured to contact a face, and comprises a left part and a right part that is symmetric to the left part, wherein each of the left part and the right part comprises:
an inside surface that is configured to contact the face, an outside surface opposite the inside surface, an upper side and a lower side,
wherein the comfort pad is configured to be detachably connectable to the frame and comprises a first flexible material and a second flexible material,
wherein the first flexible material has a chamber,
wherein the second flexible material is in the chamber,
wherein edges of the first flexible material have mating fasteners, and
wherein an average roughness of the inside surface is less than or equal to 10 micrometers.

17. The comfort pad according to claim 16, wherein each of the left part and right part has a left side and a right side opposite the left side, the left side and the right side do not extend beyond connection points of the sidebands with other components, and the other components include the frame or a headband.

18. The comfort pad according to claim 16, wherein the first flexible material includes a fabric material, and the second flexible material includes a foam material.

19. The comfort pad according to claim 16, wherein the contour of the inner surface, which is similar to the shape of the sidebands, includes one of the following shapes: straight line-shaped, curved line-shaped, Y-shaped, V-shaped, or T-shaped.

20. The comfort pad according to claim 16, wherein a part of the frame connectable to the comfort pad includes the sidebands or the forehead support.

21. The comfort pad according to claim 1, wherein the first flexible material is a fabric.

22. (canceled)

23. The comfort pad according to claim 16, wherein the mating fasteners is a zipper.
